(19) United States
(12) Patent Application Publication
(10) Pub. No.: US 2025/0304891 A1
(43) Pub. Date: Oct. 2, 2025

(54) MICROPHYSIOLOGICAL AIR-LIQUID INTERFACE SYSTEM MIMICKING PULMONARY IMMUNE RESPONSE

(71) Applicant: Seoul National University Hospital, Seoul (KR)

(72) Inventors: Young-Jae Cho, Seongnam-Si (KR); Eun-Young Eo, Seongnam-Si (KR); Sukki Cho, Seongnam-Si (KR); Insu Lee, Seongnam-Si (KR); Hyung-Jun Kim, Seongnam-Si (KR); Bokyong Kim, Seongnam-Si (KR); Jieun Kim, Seongnam-Si (KR)

(73) Assignee: Seoul National University Hospital, Seoul (KR)

(21) Appl. No.: 18/942,698

(22) Filed: Nov. 9, 2024

(30) Foreign Application Priority Data
Nov. 9, 2023 (KR) 10-2023-0154651
Jun. 19, 2024 (KR) 10-2024-0079917

(51) Int. Cl. C12M 3/00; C12M 1/00; C12M 1/12; C12N 5/071

(52) U.S. Cl. CPC C12M 21/08; C12M 25/02; C12M 29/10; C12N 5/0688; C12N 5/069; C12N 2501/999; C12N 2503/04; C12N 2506/03; C12N 2513/00

(57) ABSTRACT

The present invention relates to a lung mimicking microphysiological system including a porous membrane including lung organoids or lung organoid-derived transitional differentiated cells, macrophages, and vascular endothelial cells; and a manufacturing method thereof. More specifically, the system of the present invention has high utility as a lung immune response-mimetic air-fluid interface microphysiological system. The system of the present invention can be exposed to an external infectious agent or a drug under the same conditions as the lung in vivo, thus can conduct a wide range of research, including modeling of lung immune responses by external infectious agents and a test for therapeutic drug efficacy according to lung infectious agents, and further can also be utilized in in vitro toxicity evaluation, disease modeling, new drug development, precision medicine, and the like.

### CROSS-REFERENCE TO RELATED APPLICATION

This application claims the benefit of Korean Patent Application No. 10-2023-0154651 filed on Nov. 9, 2023 and Korean Patent Application No. 10-2024-0079917 filed on Jun. 19, 2024, the entire contents of which are incorporated herein by reference.

### BACKGROUND OF THE INVENTION

### Field of the Invention

The present invention relates to a lung mimicking microphysiological system including a porous membrane including lung organoids or lung organoid-derived transitional differentiated cells, and vascular endothelial cells; and a manufacturing method thereof.

### Description of the Related Art

The lung is an essential organ responsible for respiration and is an organ that obtains oxygen and expels carbon dioxide through inhalation and exhalation of air. Red blood cells in the blood that pass through the capillaries of the alveoli in the lung, where the gas exchanges, carry carbon dioxide produced in the body, then expel the carbon dioxide through the alveoli to the outside, take in oxygen from the atmosphere, and transport the oxygen to the entire body. Therefore, unlike other organs in a human body, the lung takes inflow and outflow of the gas.

The immune response is an essential response to damage of tissues due to external factors and recovery. In addition, the immune response is a physiological response closely related to infection prevention such as vaccines and is an important element in the development and evaluation of advanced biotechnology such as mRNA vaccines, stem cells, and gene therapy. A respiratory immune response is closely related to a series of processes from infection to onset and recovery by external infectious agents such as the recent coronavirus. The immune response is also closely related to the performance and side effects of developed vaccines and therapeutic agents and corresponds to an important research subject that is considered at all steps from the mechanism of the disease to prevention and treatment. In addition, major respiratory diseases such as pulmonary fibrosis, chronic obstructive pulmonary disease, and asthma are also closely related to abnormal immune responses. These immune responses occur in a complex environment involving various cells and factors and thus are difficult to reproduce the immune response with a simple culture dish model, and human immune research using animal models is inevitably limited due to species specificity.

As an alternative to this, research models that mimic human physiology in vitro, such as tissue chips or organoids, are emerging as new alternatives that can replace animal experiments in existing preclinical steps. The tissue chip is a system that mimics, in vitro, various physicochemical microphysiological changes such as ‘air-blood material exchange’ and ‘repetitive expansion-contraction caused by breathing’ that are the special environment of the respiratory system and is a model that reproduces the structural characteristics and functions of respiratory tissues, and the first organ chip was the lung tissue chip proposed by Wyss in the US in 2010, and the technology is currently commercialized by Emulate, Inc. in the US. Another lung organ chip is a model from Alveolix AG in Switzerland but corresponds to a structure that only mimics the epithelial-endothelial cell bilayer structure of “alveolus” from a clinical perspective.

Existing lung tissue reproducing microphysiological systems mainly utilize only the simple epithelial-endothelial cell bilayer structure of alveoli or bronchial tubes, and even in case of using immune cells, it is only possible to reproduce immune responses in a limited way due to the structure thereof.

### SUMMARY OF THE INVENTION

Example embodiments of the present invention are to provide a lung mimicking microphysiological system by physically separating alveoli and immune cells and culturing the alveoli and the immune cells to sufficiently reproduce the structure and the function of each tissue, and by enabling the movement of immune cells and related factors through blood vessels, like an actual human immune response, to be able to reproduce lung immune responses in various normal or disease states, and a manufacturing method thereof.

In one aspect, the present invention provides a lung mimicking microphysiological system that includes a porous membrane including lung organoids or lung organoid-derived transitional differentiated cells, and vascular endothelial cells.

In another aspect, the present invention provides a lung immune response-mimetic microphysiological system, comprising: an air contact part, a lung tissue mimicking part, and a body fluid perfusion mimicking part, in which the lung tissue mimicking part includes a porous membrane that includes lung organoids or lung organoid-derived transitional differentiated cells, and vascular endothelial cells, the lung organoids or the lung organoid-derived transitional differentiated cells of the porous membrane face the air contact part, and the vascular endothelial cells of the porous membrane face the body fluid perfusion mimicking part.

In another aspect, the present invention provides a method for manufacturing a lung mimicking microphysiological system, comprising: (1) coating a porous membrane with an extracellular matrix; (2) seeding and culturing lung organoids or lung organoid-derived transitional differentiated cells on one side of the coated porous membrane; and (3) seeding and culturing vascular endothelial cells on the other side of the coated porous membrane.

The system according to an aspect of the present invention can be utilized as an excellent platform as a research model for studying mechanisms of lung immune responses and lung-related diseases. Specifically, the system can be utilized for studying the mechanisms of immune responses and diseases related thereto for lungs infected with viruses such as coronaviruses or bacteria such as non-tuberculous mycobacteria. In addition, since the system can replace existing cells and animal models in the non-clinical research step, there is no animal ethics issue, and research costs can be reduced.

The system according to an aspect of the present invention can solve problems due to differences between donors or species by including lung organoids or lung organoid-derived transitional differentiated cells and can mimic lung immune responses more similar to lung immune responses of a human body by undergoing air-liquid interface (ALI) culture for a certain period of time.

By the system according to an aspect of the present invention, a wide range of research including implementation of pulmonary disease models, a test for therapeutic drug efficacy, and a test for other harmful substances can be conducted, and further, in vitro diagnosis and personalized medicine prescription can be performed.

### BRIEF DESCRIPTION OF THE DRAWINGS

The above and other aspects, features and advantages of the disclosed example embodiments will be more apparent from the following detailed description taken in conjunction with the accompanying drawings in which:

FIG. 1 is a conceptual diagram of a lung mimicking microphysiological system in case of including lung organoid-derived transitional differentiated cells according to an embodiment of the present invention;

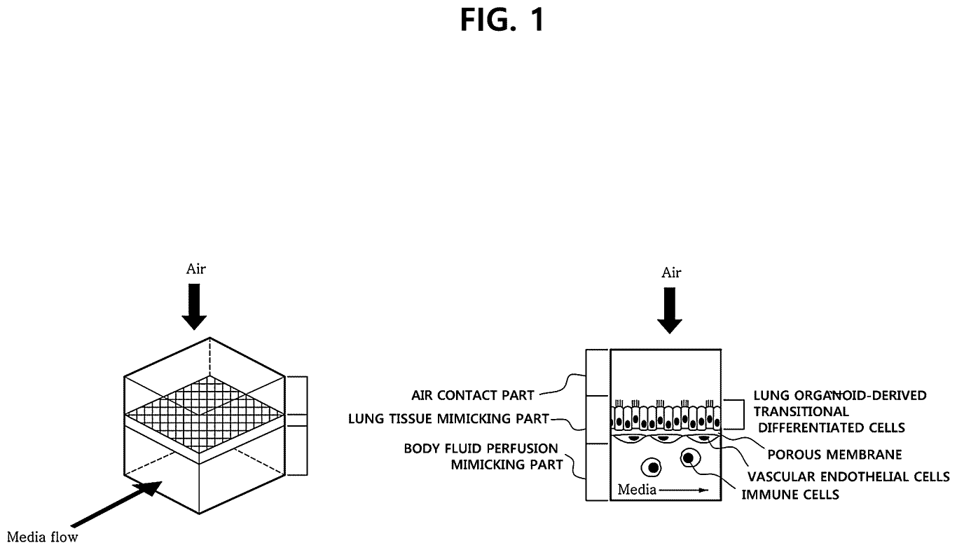

FIG. 2 is a conceptual diagram of a lung mimicking microphysiological system in case of including lung organoids according to an embodiment of the present invention;

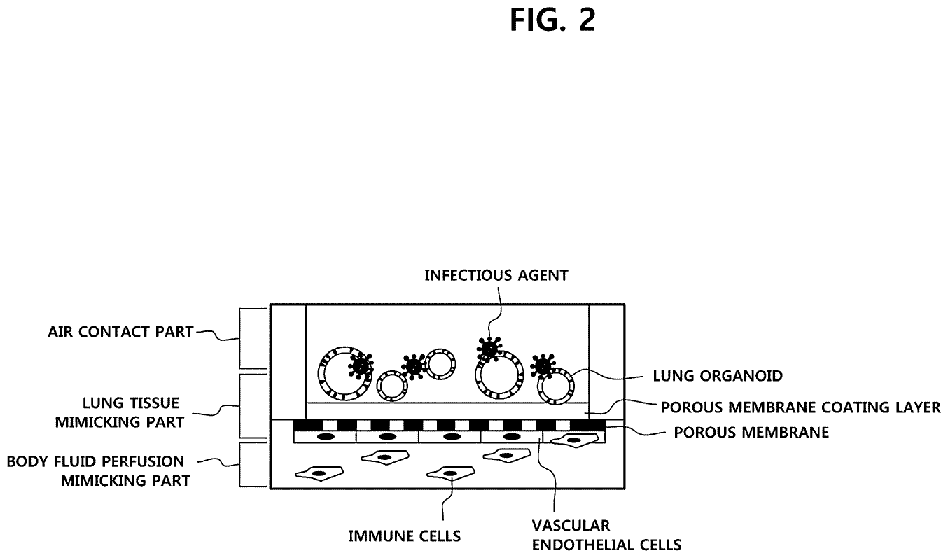

FIG. 3 is images illustrating a conceptual diagram of a lung mimicking microphysiological system according to an embodiment of the present invention and an example of a use thereof;

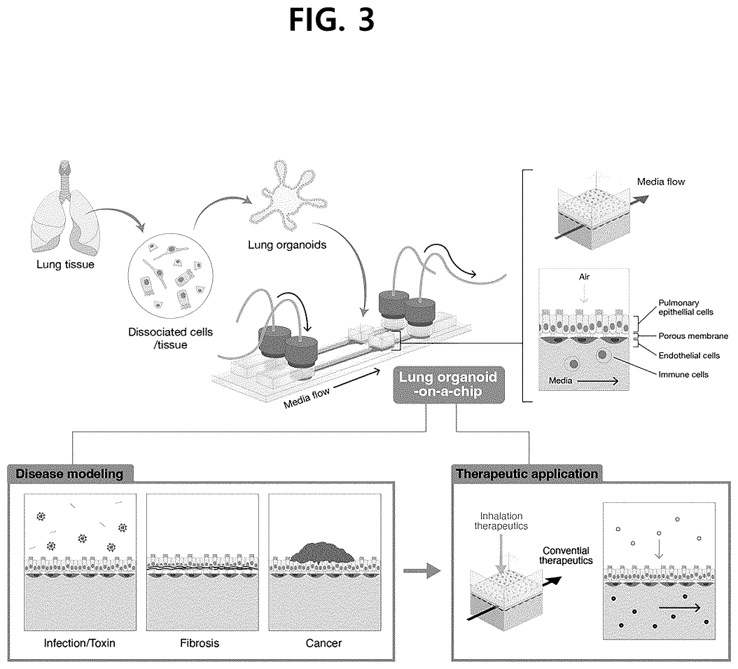

FIG. 4 illustrates results obtained by confirming the characteristics of lung organoids manufactured to apply lung organoids to a system according to an embodiment of the present invention through immunofluorescence analysis images, FACS analysis, and H & E staining;

FIG. 5 illustrates results obtained by confirming the characteristics of lung organoid-derived transitional differentiated cells according to an embodiment of the present invention through immunofluorescence analysis images;

FIG. 6 illustrates photographs of organ chips for applying a system according to an embodiment of the present invention and data obtained by measuring lengths, widths, depths, and volumes of the respective structures;

FIG. 7 is a mimetic diagram of various organoid cultures utilizing an organ chip for applying a system according to an embodiment of the present invention;

FIG. 8 is a mimetic diagram of a case where lung organoids are cultured (for 10 days or more) in a system according to an embodiment of the present invention and infected with coronavirus;

FIG. 9 shows IFA analysis results of organoid-3D models seeded and cultured in a system according to an embodiment of the present invention in case of being not infected with a virus (control group) and in case of being infected with SARS-COV-2-RVP (respiratory virus panel);

FIG. 10 shows IFA analysis results of organoid-3D models seeded and cultured in a system according to an embodiment of the present invention in a control group, in case of being infected with SARS-COV-2 (Wuhan, Delta, Omicron), and in case of being treated with macrophages;

FIG. 11 illustrates a mimetic diagram of lung organoid-derived transitional differentiated (TD) cells in a system according to an embodiment of the present invention in case of being cultured (for 20 days or more) and infected with a coronavirus;

FIG. 12 shows IFA analysis results of lung organoid-derived TD cells seeded and cultured in a system according to an embodiment of the present invention in case of being not infected with a virus (control group) and in case of being infected with SARS-COV-2-RVP (respiratory virus panel);

FIG. 13 shows IFA analysis results of lung organoid-derived TD cells seeded and cultured in a system according to an embodiment of the present invention in case of being infected with SARS-COV-2-RVP and then seeding and culturing macrophages;

FIG. 14 is a mimetic diagram of lung organoid-derived TD cells cultured in a system according to an embodiment of the present invention in case of proceeding ALI culture (for 28 days or more in total) and then infecting with a coronavirus;

FIG. 15 shows IFA analysis results of lung organoid-derived TD cells seeded and cultured in a system according to an embodiment of the present invention in case of being not infected with any virus (control group) after ALI culture and in case of being infected with SARS-COV-2-RVP after ALI culture;

FIG. 16 is a graph showing IL-6 expression levels according to three culture conditions of lung organoids and organoid-derived TD cells seeded and cultured in a system according to an embodiment of the present invention;

FIG. 17 is a modeling mimetic diagram of a system according to an embodiment of the present invention in case of being infected with non-tuberculosis mycobacteria (NTM);

FIG. 18 shows IFA analysis results of each model in a case where a system according to an embodiment of the present invention is infected with non-tuberculosis mycobacteria; and

FIG. 19 is IL-6 ELISA analysis results for each model in a case where a system according to an embodiment of the present invention is infected with non-tuberculosis mycobacteria.

### DETAILED DESCRIPTION OF THE PREFERRED EMBODIMENTS

Hereinafter, exemplary implementations of the present invention are described in more detail.

The embodiments of the present invention disclosed in the description are merely exemplified for the purpose of explanation, and the embodiments of the present invention may be implemented in various forms and should not be construed as being limited to the embodiments described in the description. In the description, details of features and techniques may be omitted to more clearly disclose example embodiments.

The present invention may be modified in various ways and may have various forms, and thus the embodiments are not intended to limit the present invention to a specific disclosed form but should be construed to include all modifications, equivalents, and substitutes included in the spirit and technical range of the present invention.

The terminology used herein is for the purpose of describing particular embodiments only and is not intended to be limiting. As used herein, the singular forms “a,” “an” and “the” are intended to include the plural forms as well, unless the context clearly indicates otherwise. Furthermore, the use of the terms a, an, etc. does not denote a limitation of quantity, but rather denotes the presence of at least one of the referenced items.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein. All methods described herein can be performed in a suitable order unless otherwise indicated herein or otherwise clearly contradicted by context. The use of any and all examples, or exemplary language (e.g., “such as”), is intended merely to better illustrate the example embodiments and does not pose a limitation on the scope of the present disclosure unless otherwise claimed. No language in the specification should be construed as indicating any non-claimed element as essential to the practice of the present disclosure as used herein.

In this application, the terms “include” or “have” and the like are intended to specify the presence of a feature, number, a step, an operation, a component, a part, or a combination thereof described in the specification but should be construed that the presence or absence of one or more other features, numbers, steps, operations, components, parts, or combinations thereof or the possibility of adding the same is not precluded in advance.

In an aspect of the present invention, the term “fluid” refers to a fine fluid perfusing inside a subject. The fluid may specifically refer to a fluid perfusing human lungs, may more specifically refer to a gas perfusing human lungs, for example, oxygen or carbon dioxide, and may be blood, lymph, or the like perfusing in blood vessels that form human lungs, and a fluid other than a gas such as blood may be replaced with a fluid containing a culture medium in vitro.

According to an aspect of the present invention, the term “organoid” refers to a three-dimensional cell aggregate formed through self-renewal, differentiation, and self-organization from adult stem cells (ASCs), embryonic stem cells (ESCs), or induced pluripotent stem cells (iPSCs).

According to an aspect of the present invention, the term “lung organoid-derived transitional differentiated cells” refers to epithelial cells differentiated from lung organoids.

According to an aspect of the present invention, the terms “part,” “module,” “device,” “system,” and the like may refer to not only hardware but also a combination with software driven thereby.

According to an aspect of the present invention, example embodiments of the present invention provide a lung mimicking microphysiological system that includes a porous membrane including lung organoids or lung organoid-derived transitional differentiated cells, and vascular endothelial cells.

The term “including” used in the “porous membrane including lung organoids or lung organoid-derived transitional differentiated cells, and vascular endothelial cells” refers to a concept that includes treating, supporting, connecting, or seeding and culturing of the lung organoids, the lung organoid-derived transitional differentiated cells, and the vascular endothelial cells on a part or all of the outside or the inside of the porous membrane, and that includes a state that can be formed by performing, without limitation, all method for culturing organoids or cells in the porous membrane by methods commonly used in the art.

According to an aspect of the present invention, the lung mimicking microphysiological system may be a lung immune response-mimetic microphysiological system.

According to an aspect of the present invention, the lung immune response-mimetic microphysiological system includes an air contact part, lung tissue mimicking part, and a body fluid perfusion mimicking part, in which the lung tissue mimicking part includes a porous membrane including lung organoids or lung organoid-derived transitional differentiated cells, and vascular endothelial cells, the lung organoids or the lung organoid-derived transitional differentiated cells of the porous membrane face the air contact part, and the vascular endothelial cells of the porous membrane face the body fluid perfusion mimicking part.

According to an aspect of the present invention, the air contact part reproduces a part where the infectious agent is exposed in the lung immune response and is characterized in that the infectious agent is exposed.

The lung tissue mimicking part reproduces the lung tissue in the lung immune response. In addition, the lung tissue may include a concept of including blood vessels surrounding the lung tissue.

The body fluid perfusion mimicking part has a function of reproducing the inside of the blood vessels around the lung tissues at the time of mimicking the lung immune response.

Through the air contact part in the system according to an example of the present invention, the lung organoids or the lung organoid-derived transitional differentiated cells are exposed to viruses or bacteria to be infected with the viruses or bacteria, so it is possible to mimic a response very similar to the actual lung infection response of the human. In addition, through the lung tissue mimicking part and the body fluid perfusion mimicking part according to an example of the present invention, it is possible to mimic a response very similar to the actual lung immune response of a human obtained by exposure to the infectious agent.

According to an aspect of the present invention, the infectious agent may be one or more selected from the group consisting of chemical substances, viruses, bacteria, and fungus. For example, the chemical substance may include fine dust or a chemical gas that induces a respiratory toxicity response. For example, the virus may be respiratory syncytial virus (RS virus), parainfluenza virus, adenovirus, influenza A and B viruses, varicella-zoster virus, measles, coronavirus, new coronavirus such as COVID-19, or severe acute respiratory syndrome coronavirus-2 (SARS-COV-2). When the virus is SARS-COV-2, the virus may include all variant viruses such as delta variant virus and omicron variant virus. For example, the bacteria may be mycobacterium tuberculosis or non-tuberculous mycobacteria.

According to an aspect of the present invention, the air contact part may be in a form of an open-top chamber. In case of the open-top chamber, the upper part is exposed to the air, and thus it is easy to mimic the air-fluid interface of the actual lung environment.

According to an aspect of the present invention, the lung tissue mimicking part or the body fluid perfusion mimicking part may further include immune cells. The immune cell may include, without limitation, cells that recognize antigens and directly or indirectly attack the antigens. For example, the immune cells may be macrophages.

The position of the immune cells may be appropriately selected from the standpoint of an ordinary person skilled in the art depending on the design of the system according to an embodiment of the present invention. For example, when the immune cells are positioned above the porous membrane, the immune cells may be mixed with the lung organoids or may be attached to the top part of a cell layer including pulmonary epithelial cells differentiated from the lung organoids. In addition, when the immune cells are positioned below the porous membrane, the immune cells may be mixed with the vascular endothelial cells, may be attached below the vascular endothelial cell layer, or may be floating in the body fluid perfusion mimicking part. In addition, the type of the immune cells may be appropriately selected from the standpoint of an ordinary person skilled in the art depending on the design of the system according to an example of the present invention, and for example, the immune cells may be macrophages.

According to an aspect of the present invention, the lung tissue mimicking part may form an air-fluid interface. The air-fluid interface may be formed by containing the air by the air contact part positioned above the lung tissue mimicking part and by containing the fluid by the body fluid perfusion mimicking part positioned below the lung tissue mimicking part. When the lung tissue mimicking part forms the air-fluid interface, ALI (air liquid interface) culture can be performed, differentiation of the lung organoids or the lung organoid-derived transitional differentiated cells is induced by the culture, and thus the lung cell configuration similar to human-derived lung tissues can be implemented, thereby more precisely mimicking the lung immune response.

According to an aspect of the present invention, by the design of the system, the lung tissue mimicking part can also form the fluid-fluid interface and thus can be appropriately selected according to the needs of a person skilled in the art.

According to an aspect of the present invention, the body fluid perfusion mimicking part may be in a form of a channel for fluid perfusion.

According to an aspect of the present invention, the air contact part is in a form of an open-top chamber, and the body fluid perfusion mimicking part is in a form of a channel for fluid perfusion.

According to an aspect of the present invention, the lung tissue mimicking part may include, without limitation, a material for helping the survival or the growth of lung organoids or lung organoid-derived transitional differentiated cells from the standpoint of an ordinary person skilled in the art. For example, the lung tissue mimicking part may further include a hydrogel but is not limited thereto.

According to an aspect of the present invention, the lung organoids may be derived from adult tissue-derived stem cells. The adult tissue-derived stem cells refer to a population of undifferentiated cells that can be differentiated into a specific cell type existing in adult tissues and can be self-renewed.

According to an aspect of the present invention, the adult tissues may be derived from isolated human lung tissues.

According to an aspect of the present invention, the lung organoid-derived transitional differentiated cells may be lung organoid-derived alveolar epithelial cells.

According to an aspect of the present invention, a material forming the porous membrane may be a polymer, and the polymer may be at least one selected from the group consisting of polyethylene terephthalate (PET), polydimethylsiloxane (PDMS), polycarprolactone (PCL), and nanofiber, but is not limited thereto. The thickness of the porous membrane may be 3 to 24 μm, specifically, may be 3 μm or more, 4 μm or more, 6 μm or more, 7 μm or more, 8 μm or more, 9 μm or more, 10 μm or more, 11 μm or more, 12 μm or more, 14 μm or more, 16 μm or more, 18 μm or more, 20 μm or more, or 22 μm or more, and may be 24 μm or less, 22 μm or less, 20 μm or less, 18 μm or less, 16 μm or less, 14 μm or less, 12 μm or less, 11 μm or less, 10 μm or less, 9 μm or less, 8 μm or less, 7 μm or less, 6 μm or less, or 4 μm or less. However, the thickness is not limited thereto and may vary depending on the size of the porous membrane in the lung immune response-mimetic microphysiological system. The pore size of the porous membrane may be 1 to 16 μm, specifically may be 1 μm or more, 2 μm or more, 3 μm or more, 4 μm or more, 5 μm or more, 6 μm or more, 7 μm or more, 8 μm or more, 9 μm or more, 10 μm or more, 11 μm or more, 12 μm or more, 13 μm or more, 14 μm or more, or 15 μm or more, and may be 16 μm or less, 15 μm or less, 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, or 2 μm or less. However, the pore size is not limited thereto and may vary depending on the porous membrane in the lung immune response-mimetic microphysiological system. The porous membrane may be coated with an extracellular matrix (ECM) so that lung organoids, lung organoid-derived transitional differentiated cells, vascular endothelial cells, or immune cells can be attached to the porous membrane. The extracellular matrix may be at least one selected from the group consisting of laminin, collagen type I, collagen type II, collagen type III, collagen type IV, collagen type V, collagen type VI, integrin, entectin, fibronectin, elastin, proteoglycan, vitronectin, poly-D-lysine, polysaccharide, and gelatin. However, the extracellular matrix is not limited thereto, as long as the extracellular matrix can cause the lung organoids, the lung organoid-derived transitional differentiated cells, the vascular endothelial cell, or the immune cells to be attached to the porous membrane.

According to an aspect of the present invention, the lung organoids or the lung organoid-derived transitional differentiated cells may be in connection with the air contained in the air contact part.

According to an aspect of the present invention, the vascular endothelial cells may be in connection with the fluid in the body fluid perfusion mimicking part.

According to another aspect of the present invention, example embodiments of the present invention provide a method for manufacturing a lung mimicking microphysiological system including: (1) coating a porous membrane with an extracellular matrix; (2) seeding and culturing lung organoids or lung organoid-derived transitional differentiated cells on one side of the coated porous membrane; and (3) seeding and culturing vascular endothelial cells on the other side of the coated porous membrane.

The description of the porous membrane, the extracellular matrix, the lung organoid or lung organoid-derived transitional differentiated cells, the vascular endothelial cells, and the lung mimicking microphysiological system is as described above.

According to an aspect of the present invention, the lung mimicking microphysiological system may be a lung immune response-mimetic microphysiological system. The description of the lung immune response-mimetic microphysiological system is as described above. According to an aspect of the present invention, seeding and culturing immune cells on any one side of the porous membrane may be further included after the step (3).

According to an aspect of the present invention, inducing lung organoids in a lung tissue sample isolated from a human may be further included before the step (2). The method for inducing lung organoids from a lung tissue sample may include, without limitation, all methods that can induce lung organoids from lung tissues from the standpoint of an ordinary person skilled in the art. The isolation may be performed including, without limitation, all methods that can isolate lung cells from the lung tissues from the standpoint of an ordinary person skilled in the art.

According to an aspect of the present invention, the inducing may include culturing lung tissues sample in a culture medium including a Rho-associated protein kinase (ROCK) inhibitor for one to four days. The ROCK inhibitor may include Y-27632 but the embodiment is not limited thereto. The culture in a culture medium including a ROCK inhibitor may be performed for one day or more, 1.1 days or more, 1.2 days or more, 1.3 days or more, 1.4 days or more, 1.5 days or more, 1.6 days or more, 1.7 days or more, 1.8 days or more, 1.9 days or more, or two or more days and may be performed for four days or less, 3.9 days or less, 3.8 days or less, 3.7 days or less, 3.6 days or less, 3.5 days or less, 3.4 days or less, 3.3 days or less, 3.2 days or less, 3.1 days or less, or three days or less. However, the embodiment is not limited thereto. The culture medium including a ROCK inhibitor may further include a material that is required for inducing lung organoids from the standpoint of an ordinary person skilled in the art. For example, the culture medium may further include Matrigel.

According to an aspect of the present invention, the lung organoid-derived transitional differentiated cells are obtained by culturing fragmented lung organoids for five to sixteen days, and the fragmentation may be treating lung organoids with a protease for one to twenty minutes. The period for culturing fragmented lung organoids may be five days or more, 5.2 days or more, 5.4 days or more, 5.6 days or more, 5.8 days or more, 6 days or more, 6.2 days or more, 6.4 days or more, 6.6 days or more, 6.8 days or more, or seven days or more and may be 14 days or less, 13.8 days or less, 13.6 days or less, 13.4 days or less, 13.2 days or less, 13 days or less, 12.8 days or less, 12.6 days or less, 12.4 days or less, 12.2 days or less, or twelve days or less. However, the present embodiment is not limited thereto. The protease may be TrypLE™, but the present embodiment is not limited thereto. The period of time for treating a protease may be one minute or more, 1.5 minutes or more, two minutes or more, 2.5 minutes or more, three minutes or more, 3.5 minutes or more, four minutes or more, 4.5 minutes or more, or five minutes or more and may be twenty minutes or less, nineteen minutes or less, 18.5 minutes or less, eighteen minutes or less, 17.5 minutes or less, seventeen minutes or less, 16.5 minutes or less, sixteen minutes or less, 15.5 minutes or less, or fifteen minutes or less. However, the present embodiment is not limited thereto. Further, the culture of fragmented lung organoids may be performed by being seeded in a medium including Matrigel, but the present embodiment is not limited thereto.

According to an aspect of the present invention, when the cells seeded in the step (2) are the lung organoid-derived transitional differentiated cells, the culturing in the step (2) may include air-liquid interface culture for two to six weeks. For example, the air-liquid interface culture may be performed for two to six weeks, 16 to 40 days, 18 to 38 days, 20 to 36 days, 22 to 34 days, 24 to 34 days, or 26 to 32 days and is preferably performed for 24 to 34 days, or 26 to 32 days.

In an example embodiment of the present invention, the culturing in step (2) is proceeded for four weeks, and a cell experiment was performed for seven days while maintaining the ALI culture state after the culture of the seeded cells was completed.

Hereinafter, the present invention is described in more detail with reference to examples. These examples are only for illustrating the present invention, and it will be apparent to an ordinary person skilled in the art that the scope of the present invention is not to be construed as being limited by these examples.

### EXAMPLES

### Manufacturing Example

### 1. Manufacturing of Lung Organoids

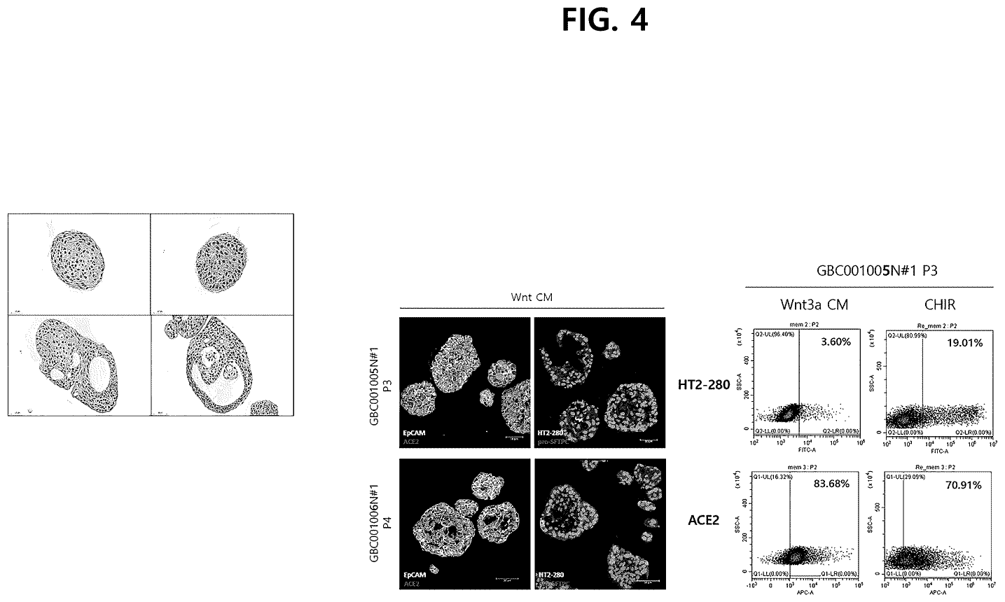

Patient-derived lung tissues were decomposed by chemical and physical methods to isolate cells. In order to isolate cells from the tissues, the tissues were treated with collagenase and then passed through a 100-um cell strainer. The obtained cells were placed in Matrigel and cultured in a 24-well plate. During the initial two to three days of culture, the cells were cultured by including a ROCK inhibitor (Y-27632) in the lung organoid culture medium. In order to confirm the cultured lung organoids, the expression of specific markers of lung organoids was confirmed by fluorescence imaging or FACS analysis (FIG. 4).

### 2. Culture of Lung Organoid-Derived Transitional Differentiated Cells

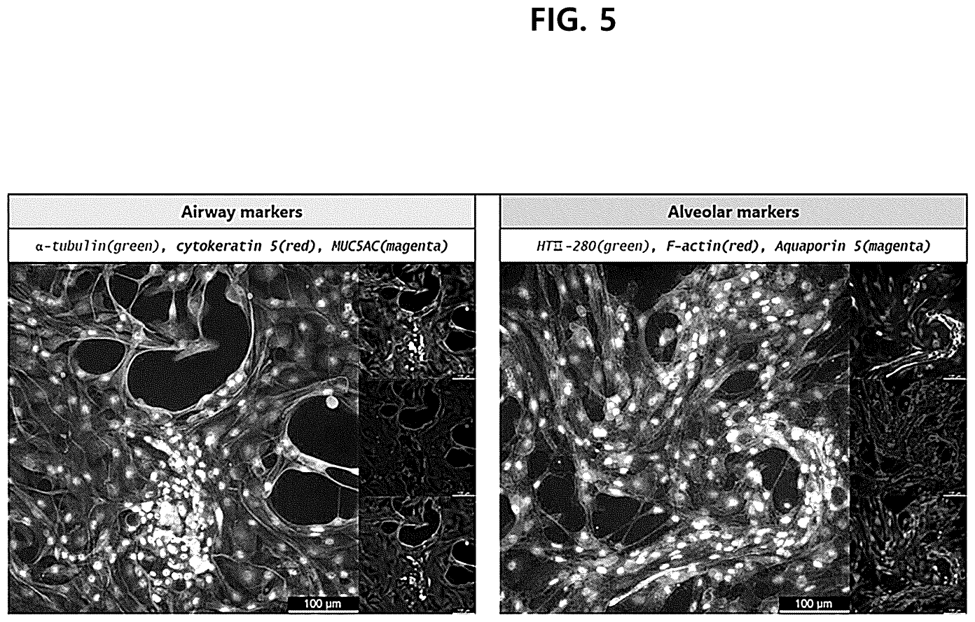

In order to perform two-dimensional cell culture (2D cell culture, a form in which cells were cultured by being attached to the bottom) by using cultured lung organoids, Matrigel was removed from the lung organoids, and the lung organoids were treated with TrypLE™ for five to fifteen minutes to fragment the lung organoids. The fragmented lung organoids were seeded in a well plate coated with Matrigel and were cultured for seven to fourteen days to culture lung organoid-derived transitional differentiated cells, and the expression of specific markers of lung organoids was confirmed by fluorescence imaging (FIG. 5).

### 3. Manufacturing of Lung Mimicking Microphysiological System

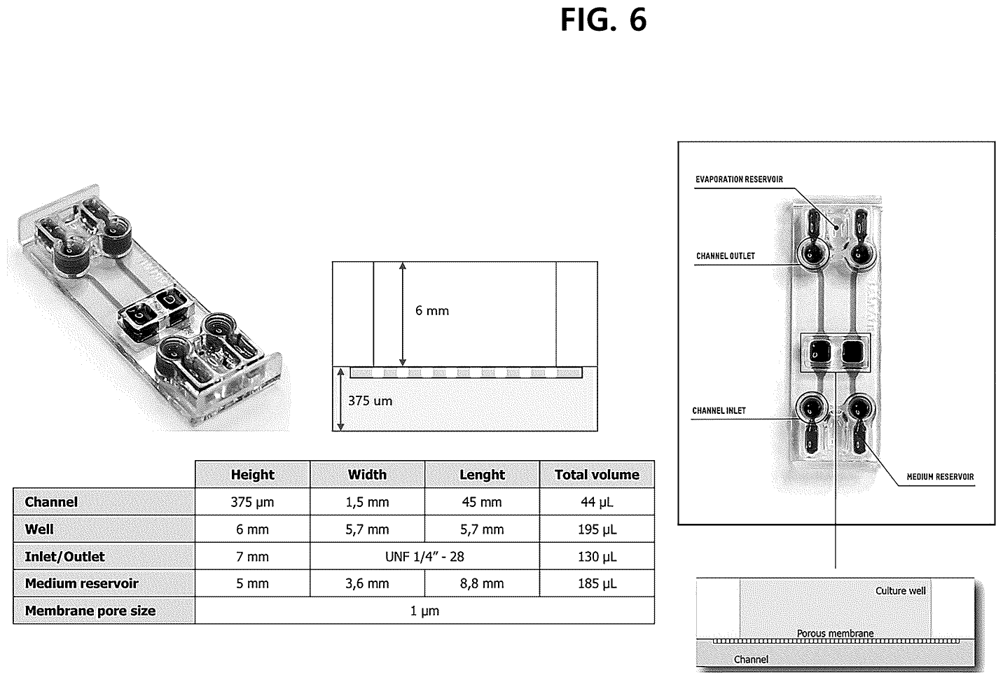

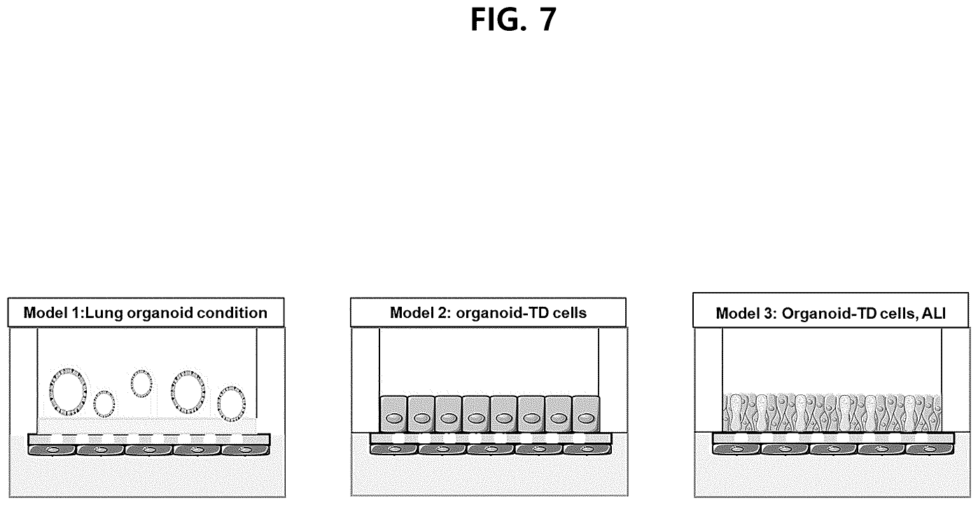

In the lung mimicking microphysiological system using the lung organoids and the lung organoid-derived transitional differentiated cells, the BE-Transflow chip of BEOnChip was used as illustrated in FIG. 6. The chip used is configured with chambers and channels and has a structure suitable for ALI culture. Using the lung organoids and the lung organoid-derived cells, it was attempted to find a lung microphysiological system similar to the human body through three culture methods, Model 1: Lung Organoids, Model 2: Lung Organoid-Derived Transitional Differentiated Cells, and Model 3: Lung Organoid-Derived Transitional Differentiated Cells and ALI culture, as illustrated in FIG. 7.

### [Experiment Example 1] Confirmation of Culture Method for Establishing Lung Microphysiological System According to Coronavirus Infection

### 1. Evaluation of Microphysiological System Using Lung Organoids

### 1-1. Confirmation of Coronavirus Infection

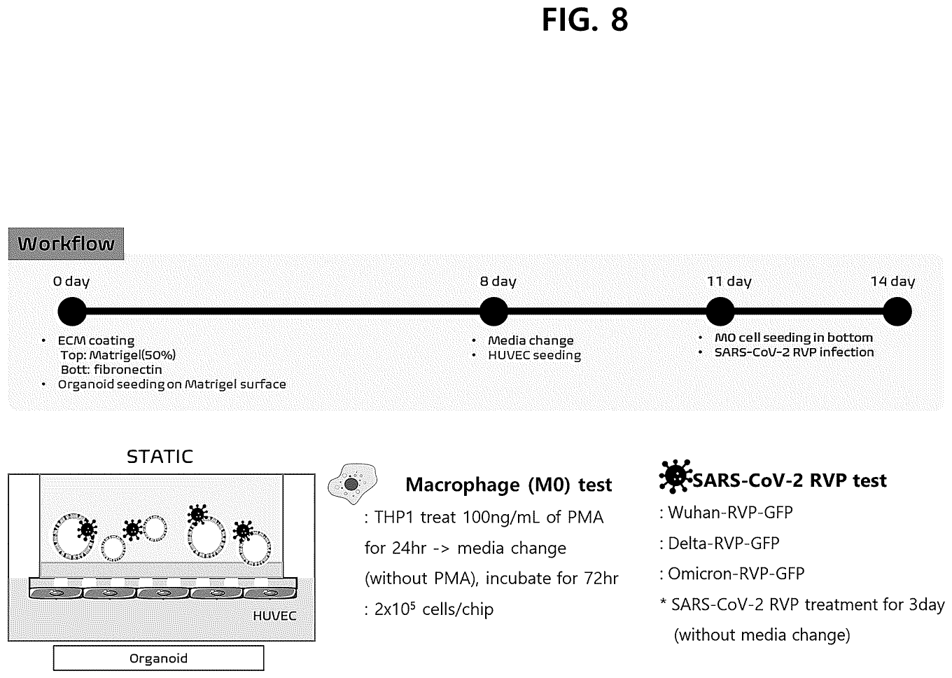

The lung organoids were cultured in Matrigel for seven to twelve days, then were seeded on a top part (chamber) of the BE-transflow chip in a state in which the Matrigel was removed, and were stabilized in the chip for seven to eight days. After that, vascular endothelial cells were seeded in a bottom part (channel) of the chip and stabilized for three to four days. For the coronavirus infection experiment, the top part of the chip where lung organoids were seeded was treated with SARS-COV-2 RVP for three days (FIG. 8).

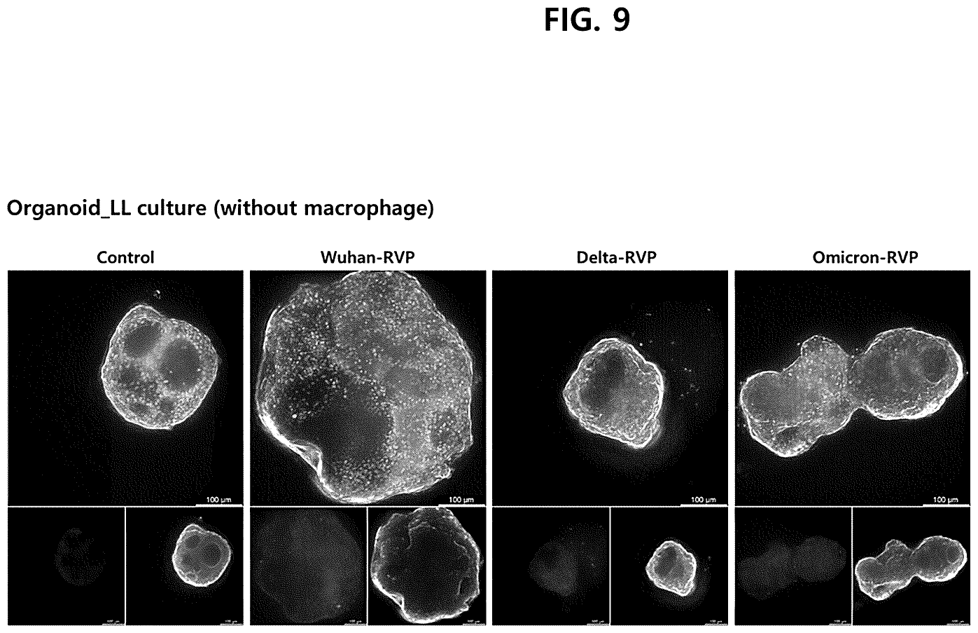

Infection was weakly confirmed in the experimental groups infected with coronavirus with respect to the control group that was not infected with coronavirus (FIG. 9, green part).

### 1-2. Confirmation of Degree of Infection According to Macrophage Seeding

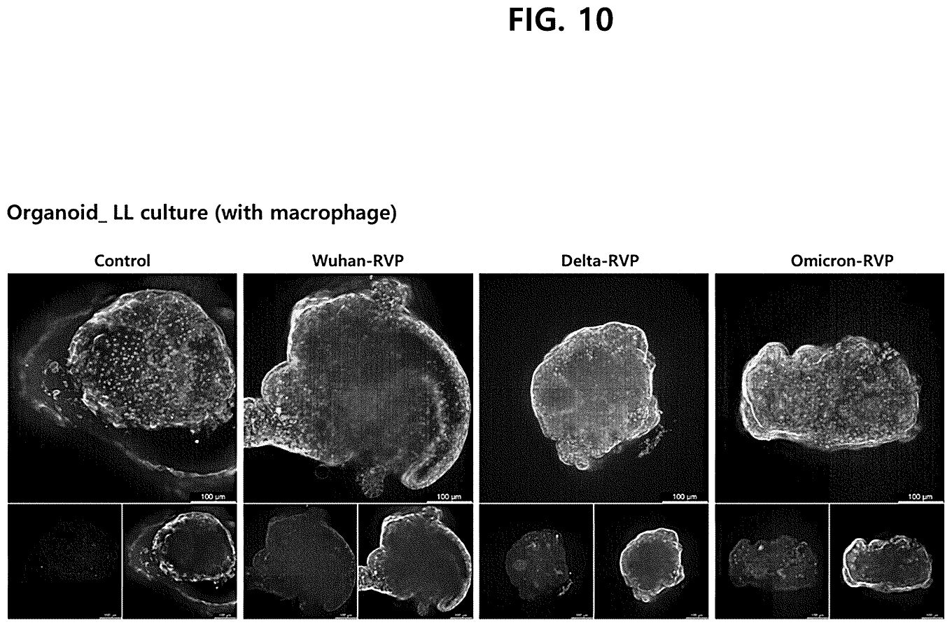

When macrophages were seeded in the bottom part of the chip, coronavirus infection was confirmed to be higher (FIG. 10, green part) than that in the experimental groups in which macrophages were not seeded (FIG. 9 above).

In the lung organoids (Model 1), it was confirmed that infection with coronavirus (SARS-COV-2 RVP) can be reproduced, but the infection was not limited to the lung organoids but was also confirmed outside of the lung organoids (in the extracellular matrix (ECM) in the top part of the chip), and thus it was difficult to clearly identify a difference from the control group. Therefore, it was confirmed that the lung organoids (Model 1) were not suitable as a culture method for establishing a lung microphysiological system using lung organoids.

### 2. Evaluation of Microphysiological System Using Lung Organoid-Derived Transitional Differentiated Cells

### 2-1. Confirmation of Coronavirus Infection

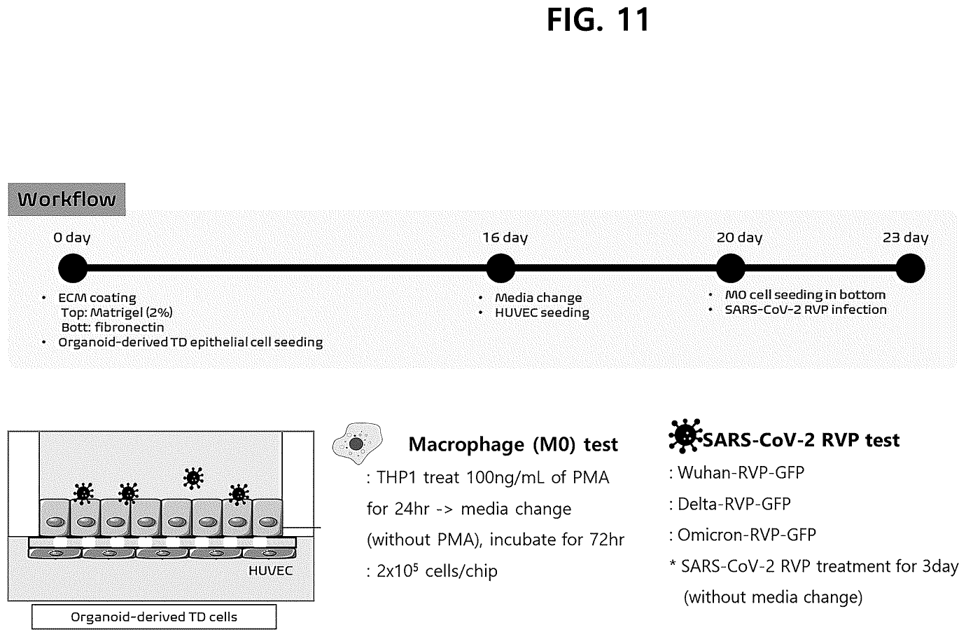

The lung organoid-derived transitional differentiated cells were seeded in the top part of the chip and stabilized for fourteen to sixteen days, and the vascular endothelial cells were seeded in the bottom part of the chip. On the fourth day from the seeding of the vascular endothelial cells, the top part of the chip was treated with coronavirus (SARS-COV-2 RVP) for three days (FIG. 11).

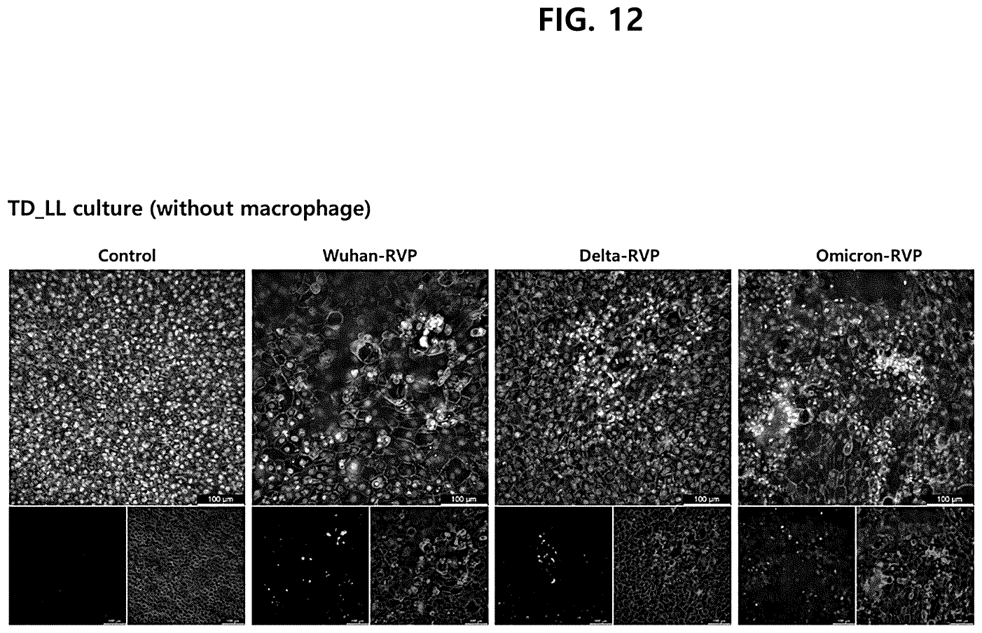

In a model using the lung organoid-derived transitional differentiated cells (Model 2), virus infection was confirmed in the experimental groups treated with coronavirus (FIG. 12).

### 2-2. Confirmation of Degree of Infection According to Macrophage Seeding

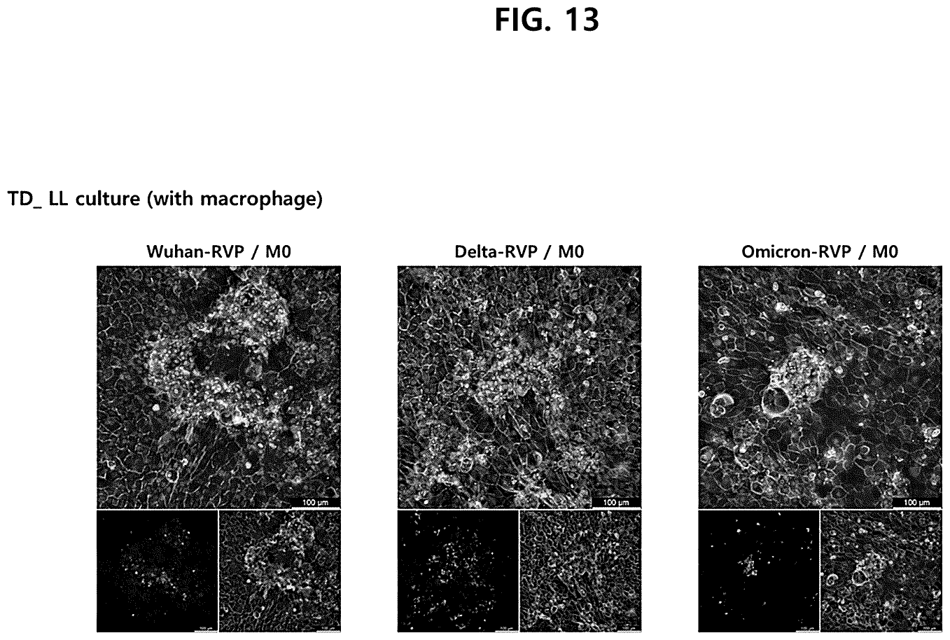

In the experimental groups in which macrophages were seeded in the bottom part of the chip, it was confirmed that the coronavirus infection was higher (FIG. 13) than that in the experimental groups in which macrophages were not seeded (FIG. 12 above).

### 2-3. Confirmation of Degree of Infection According to Presence or Absence of Ali Culture

The lung organoid-derived transitional differentiated cells were seeded in the top part of the chip, and after seven to eight days, ALI culture was proceeded for four weeks (Model 3).

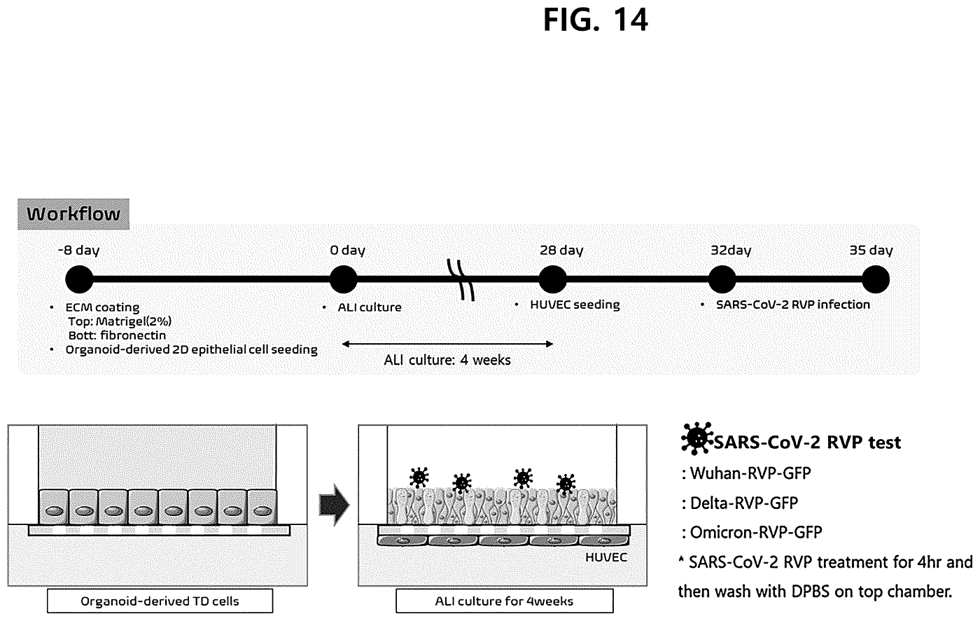

On the fourth week (Day 28), the vascular endothelial cells were seeded in the bottom part of the chip, and the top part of the chip was treated with coronavirus (SARS-COV-2 RVP) after four days (FIG. 14).

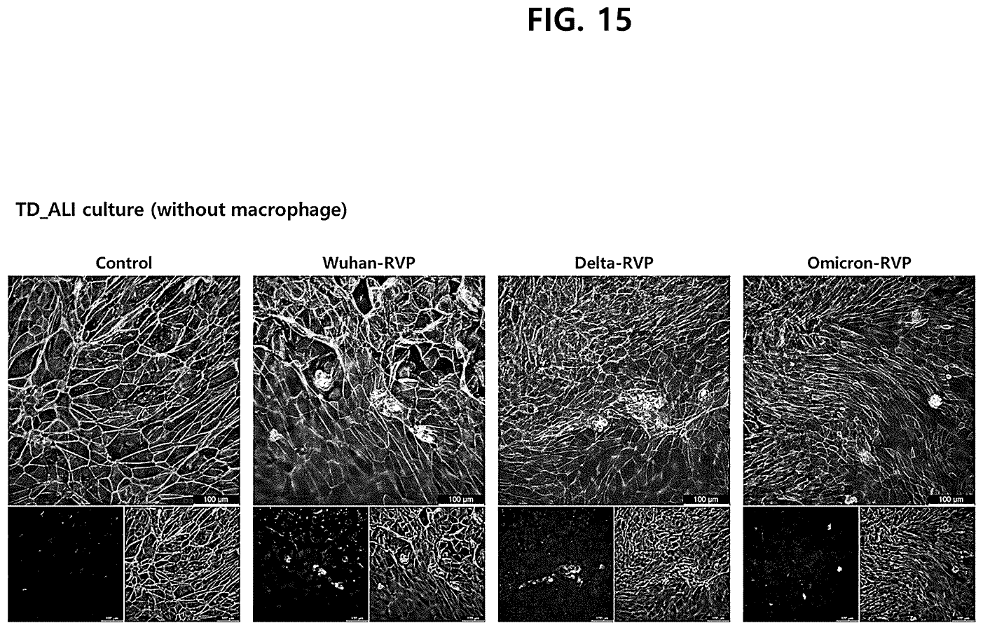

The lung organoid-derived transitional differentiated cells were ALI-cultured to differentiate the pulmonary epithelial cells, and then infection was confirmed in the experimental groups treated with coronavirus (SARS-COV-2 RVP) (FIG. 15).

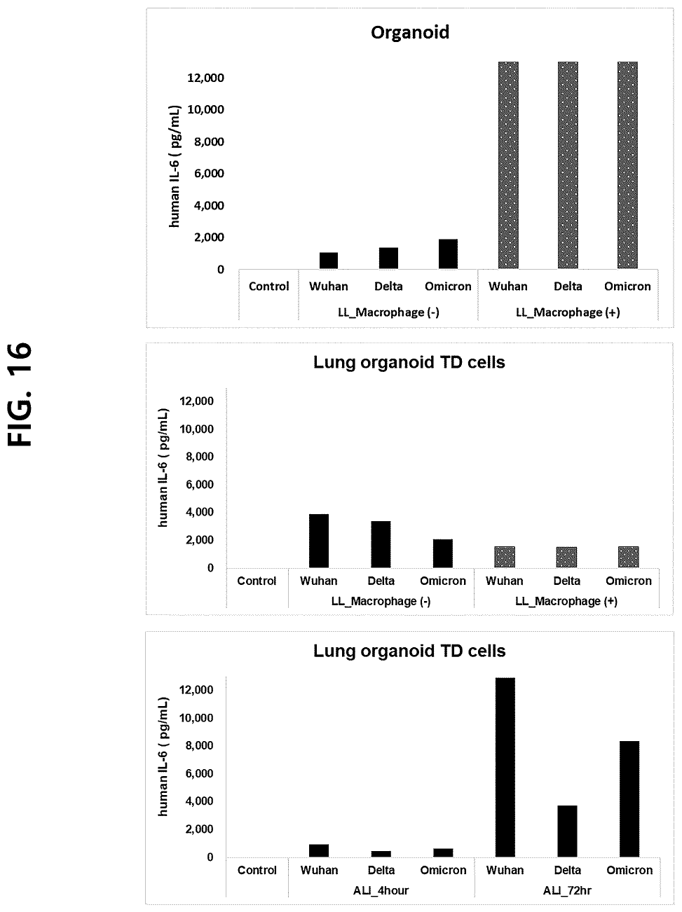

In order to confirm inflammatory responses due to coronavirus infection in the three models, expression levels of IL-6 cytokine that is an inflammatory factor in a culture medium were confirmed. In the lung organoids (Model 1) and the lung organoid-derived transitional differentiated cells and ALI culture (Model 3), it was confirmed that expression levels of IL-6 cytokine were high (FIG. 16).

In other words, in the fluorescence images of Model 2 and Model 3 using the lung organoid-derived transitional differentiated cells, it was confirmed that infection was higher in Model 2. However, it was confirmed that the substantial inflammatory responses that occurred in case of infection with coronavirus were higher in Model 3, and thus it was confirmed that the most suitable culture method for establishing a lung microphysiological system using lung organoids was Model 3.

### [Experiment Example 2] Confirmation of Lung Immune Response Mimicking Due to Non-Tuberculosis Mycobacterium Infection

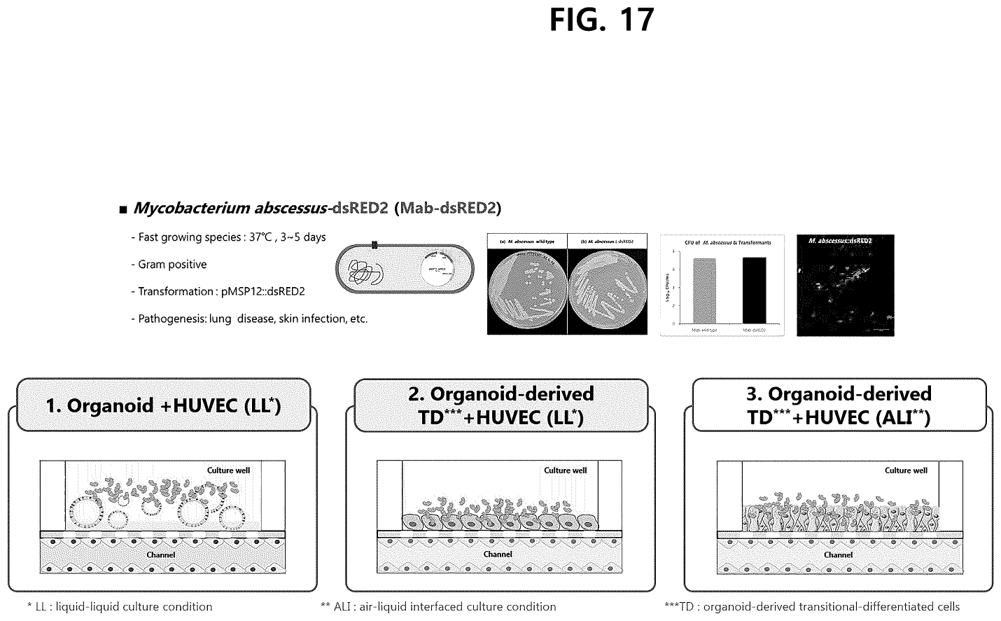

FIG. 17 is a strain that was produced by transforming Mycobacterium abscessus that is one of representative species of non-tuberculosis mycobacteria, to express dsRED2 fluorescent protein. The transformation of the strain was performed to visualize the degree of infection of the strain in the subsequent immunofluorescence assay.

The non-tuberculosis mycobacterial transformed strains were mixed with cell cultures and were treated under a co-culture condition of lung organoids and vascular cells (Model 1), a co-culture condition of lung organoid-derived transitional differentiated cells and vascular cells under liquid-liquid culture conditions (Model 2), and a co-culture condition of lung organoid-derived transitional differentiated cells and vascular cells under air-liquid culture conditions (Model 3), thereby establishing and inducing the infection models.

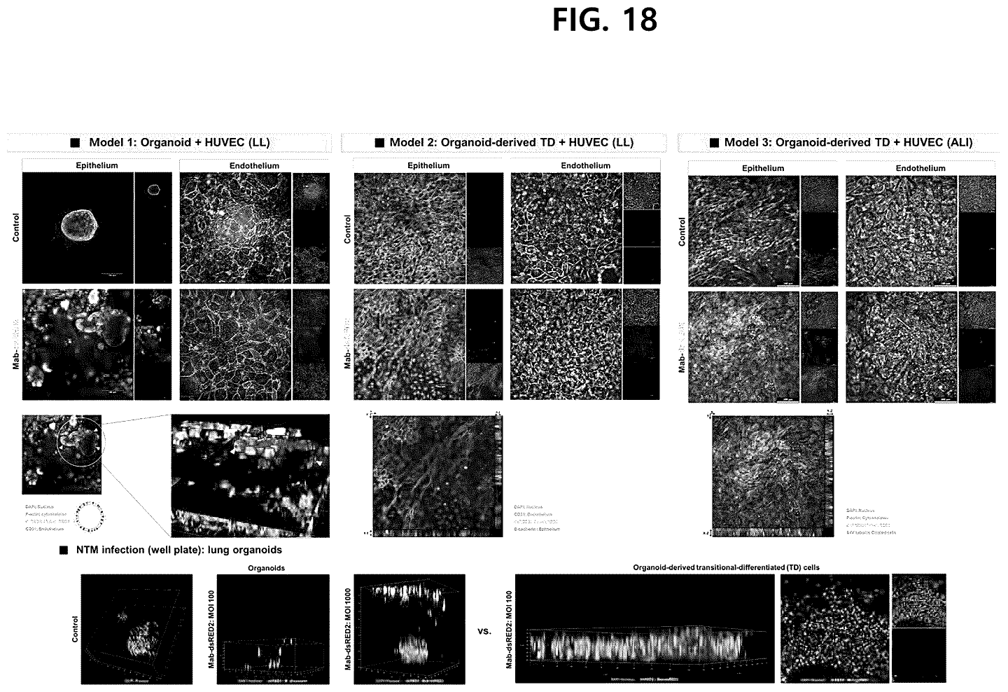

Results of the immunofluorescence assay performed for visualizing and analyzing the degree of infection of respective models corresponding to the above three conditions are illustrated in FIG. 18.

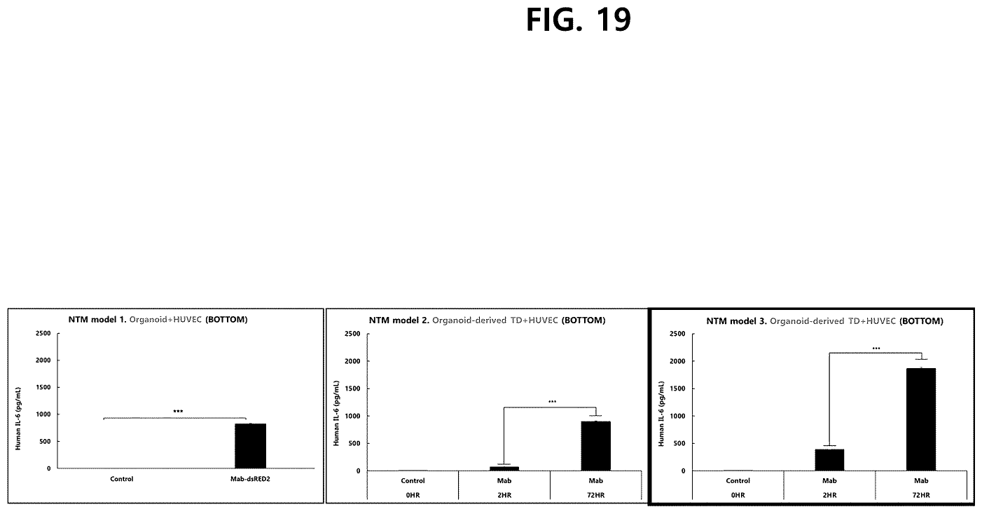

Inflammatory response marker analyses using enzyme-linked immunosorbent assay (ELISA) were proceeded, and results thereof are illustrated in FIG. 19.

As the results of the experiments, in all of Models 1 to 3, significant increases in cell damage and inflammatory cytokine levels by non-tuberculosis mycobacteria were shown, and thus their potential for use as infection models was confirmed. However, it was understood that the infection model with the most distinct response and highest utility was Model 3, which is a co-culture of lung organoid-derived transitional differentiated cells and vascular cells under the air-liquid culture condition.

While non-limiting and example embodiments of the present disclosure have been described above, the technical idea of the present disclosure is not limited to the accompanying drawings or the above description. It will be apparent to a skilled person in the art that various modifications are possible without departing from the technical ideas of the present disclosure, and that such modifications fall within the scope of the claims of the present disclosure.

This invention relates to national research and development project performed by Seoul National University Bundang Hospital where the national research and development project is of Strategic International Collaborative Research and Development (R&D) and named as ‘Study on Human Infection Control Mechanism of Novel and Unsolved Respiratory Infectious Diseases Based on Organoid-on-a-Chip Platform’, and period of the project is from Sep. 20, 2021 to Mar. 31, 2024 (assignment unique number is 11711175002, assignment number is 2021K1A4A7A02097757). Also, the project's name of Department is Ministry of Science and ICT, the project's name of Project Management (Professional) Institution is National Research Foundation of Korea.

What is claimed is:

1. A lung mimicking microphysiological system, comprising:
a porous membrane including lung organoids or lung organoid-derived transitional differentiated cells, and vascular endothelial cells.

2. The lung mimicking microphysiological system according to claim 1,
wherein the lung mimicking microphysiological system is a lung immune response-mimetic microphysiological system.

3. The lung mimicking microphysiological system according to claim 2, wherein the lung immune response-mimetic microphysiological system comprises:
an air contact part;
a lung tissue mimicking part; and
a body fluid perfusion mimicking part,
wherein the lung tissue mimicking part includes a porous membrane including lung organoids or lung organoid-derived transitional differentiated cells, and vascular endothelial cells,
the lung organoids or the lung organoid-derived transitional differentiated cells of the porous membrane face the air contact part, and
the vascular endothelial cells of the porous membrane face the body fluid perfusion mimicking part.

4. The lung mimicking microphysiological system according to claim 3,
wherein the air contact part is exposed to an infectious agent.

5. The lung mimicking microphysiological system according to claim 4,
wherein the infectious agent is at least one selected from the group consisting of chemical substances, viruses, bacteria, and fungus.

6. The lung mimicking microphysiological system according to claim 5,
wherein the viruses are severe acute respiratory syndrome coronavirus-2 (SARS-COV-2), and/or
the bacteria is non-tuberculous mycobacteria.

7. The lung mimicking microphysiological system according to claim 3,
wherein the air contact part is in a form of an open-top chamber.

8. The lung mimicking microphysiological system according to claim 3,
wherein the lung tissue mimicking part or the body fluid perfusion mimicking part further includes immune cells.

9. The lung mimicking microphysiological system according to claim 3,
wherein the lung tissue mimicking part has an air-fluid interface.

10. The lung mimicking microphysiological system according to claim 3,
wherein the body fluid perfusion mimicking part is in a form of a channel for fluid perfusion, or
the body fluid perfusion mimicking part is in a form of a channel for fluid perfusion, and the air contact part is in a form of an open-top chamber.

11. The lung mimicking microphysiological system according to claim 1,
wherein the lung organoids are derived from adult tissue-derived stem cells, or
the lung organoids are derived from adult tissue-derived stem cells, and the adult tissue is derived from isolated human lung tissues.

12. The lung mimicking microphysiological system according to claim 1,
wherein the porous membrane is coated with an extracellular matrix (ECM), or
the porous membrane is coated with an extracellular matrix (ECM), and the extracellular matrix is at least one selected from the group consisting of laminin, collagen type I, collagen type II, collagen type III, collagen type IV, collagen type V, collagen type VI, integrin, entectin, fibronectin, elastin, proteoglycan, vitronectin, poly-D-lysine, polysaccharide, gelatin, or Matrigel.

13. The lung mimicking microphysiological system according to claim 1,
wherein the porous membrane has a pore size of 0.45 or 3 μm.

14. The lung mimicking microphysiological system according to claim 3,
wherein the lung organoids or the lung organoid-derived transitional differentiated cells are in connection with the air contained in the air contact part, and/or
the vascular endothelial cells are in connection with a fluid in the body fluid perfusion mimicking part.

15. A method for manufacturing the lung mimicking microphysiological system according to claim 1, the method comprising:
(1) coating a porous membrane with an extracellular matrix;
(2) seeding and culturing lung organoids or lung organoid-derived transitional differentiated cells on one side of the coated porous membrane; and
(3) seeding and culturing vascular endothelial cells on the other side of the coated porous membrane.

16. The method according to claim 15,
wherein the lung mimicking microphysiological system is a lung immune response-mimetic microphysiological system.

17. The method according to claim 15, further comprising:
inducing lung organoids from a lung tissue sample isolated from a human before the step (2); and/or
seeding and culturing immune cells on any one side of the porous membrane after the step (3).

18. The method according to claim 15,
wherein the inducing includes culture of the lung tissue sample for one to four days in a culture medium including a Rho-associated protein kinase (ROCK) inhibitor.

19. The method according to claim 15,
wherein the lung organoid-derived transitional differentiated cells are obtained by culturing fragmented lung organoid for five to sixteen days, and
the fragmentation is treating the lung organoid with protease for one to thirty minutes.

20. The method according to claim 15,
wherein when the cells seeded in the step (2) are lung organoid-derived transitional differentiated cells, the culturing in the step (2) includes air-liquid interface culture for two to six weeks.
